(19) United States
(12) Patent Application Publication
(10) Pub. No.: US 2025/0329455 A1
(43) Pub. Date: Oct. 23, 2025

(54) METHOD FOR PREDICTING A VITAL SIGN, VITAL SIGN MONITOR, AND METHOD FOR PRODUCING A VITAL SIGN MONITOR

(71) Applicant: ams-OSRAM AG, Premstaetten (AT)

(72) Inventors: Lina Wei, Boulogne-Billancourt (FR); Olivier Feher, Martigues (FR); Yannick Menet, Chatillon (FR)

(21) Appl. No.: 18/638,189

(22) Filed: Apr. 17, 2024

(51) Int. Cl. G16H 50/20; A61B 5/00; A61B 5/0205; G16H 10/60; G16H 50/30

(52) U.S. Cl. CPC G16H 50/20; A61B 5/02055; A61B 5/7264; G16H 10/60; G16H 50/30

(57) ABSTRACT

A method for predicting a vital sign of a person comprises recording a time series of a bio signal of a person as a first dataset, obtaining a demographic data of the person, extracting a feature vector from the first dataset using a machine-learning based feature extractor, generating an embedding vector from the demographic data using an embedding model, and predicting a vital sign of the person from the feature vector and the embedding vector using a machine-learning based regressor.

### TECHNICAL FIELD

The present invention relates to a method for predicting a vital sign of a person, to a vital sign monitor, and to method for producing a vital sign monitor.

### BACKGROUND

It is known in the state of the art to predict a vital sign of a person on the basis of a measurement of a bio signal of the person.

### SUMMARY

Embodiments provide a method for predicting a vital sign of a person. Further embodiments provide a vital sign monitor. Yet further embodiments provide a method for producing a vital sign monitor.

A method for predicting a vital sign of a person comprises recording a time series of a bio signal of a person as a first dataset, obtaining a demographic data of the person, extracting a feature vector from the first dataset using a machine-learning based feature extractor, generating an embedding vector from the demographic data using an embedding model, and predicting a vital sign of the person from the feature vector and the embedding vector using a machine-learning based regressor.

This method allows to take the demographic data of the person into account for predicting the vital sign of the person. Advantageously, this may increase the reliability of the prediction of the vital sign. The method allows to combine the first dataset, which is a time series, with the demographic data, which is constant in time. This is achieved by first extracting a feature vector from the first dataset and combining the feature vector with an embedding vector generated from the demographic data. In result, the method allows for a calibration-free prediction of the vital sign, which may allow the method to be used in consumer devices, for example.

In a variant of the method, the vital sign is a body temperature, a heart rate, a respiratory rate, or a blood pressure. These are considered the four main vital signs of a human body.

In a variant of the method, the first dataset is a photoplethysmogram or an electrocardiogram. Advantageously, such a first dataset provides information that allows a prediction of a vital sign. A photoplethysmogram may advantageously be recorded in a continuous and nondisturbing manner using optical techniques. Electrocardiograms may provide particularly informative data on the activity of a heart.

In a variant of the method, recording the first dataset is followed by preprocessing the first dataset to remove high-frequency noise or a low-frequency modulation. Advantageously, such preprocessing may improve the accuracy and reliability of the method.

In a variant of the method, the first dataset comprises 256 floating point numbers. It has shown that using a first dataset of this size allows for an efficient and reliable extraction of a feature vector.

In a variant of the method, the demographic data is a body height, a body weight, a body mass index (BMI), an age, or a gender of the person. Advantageously, this demographic data provides useful information for the calibration of the predicted vital sign.

In a variant of the method, generating the embedding vector includes categorizing the demographic data. Categorizing the demographic data allows to generate an embedding vector from the demographic data in the case that the demographic data is a continuous variable.

In a variant of the method, the embedding vector comprises between 3 and 32 elements. An embedding vector comprising such a number of elements has shown to be practical for predicting the vital sign using a regressor that takes both the feature vector and the embedding vector as inputs. If the size of the embedding vector is too small, the embedding vector might be disregarded by the regressor in the prediction of the vital sign.

In a variant of the method, at least one further demographic data of the person is obtained. At least one further embedding vector is generated from the at least one further demographic data using at least one further embedding model. The vital sign of the person is predicted from the feature vector, the embedding vector, and the at least one further embedding vector. Advantageously, using more than one demographic data may increase the accuracy and reliability of the predicted vital sign.

A vital sign monitor comprises a sensor for recording a time series of a bio signal of a person as a first dataset. The vital sign monitor is adapted for obtaining a demographic data of the person and for executing a method as described above.

This vital sign monitor may allow for predicting a vital sign with a high accuracy and reliability. Advantageously, this vital sign monitor may take the demographic data of the person into account in the prediction of the vital sign. The vital sign monitor may be integrated into a portable device, such as watch, for example.

Some variants of the vital sign monitor comprise a data memory for storing the demographic data. Advantageously, this allows the vital sign monitor to use the demographic data repeatedly for predicting vital signs.

A method for producing a vital sign monitor comprises providing a training dataset having a plurality of data records. Each data record comprises a time series of a bio signal of a person as a first dataset, a demographic data of the person, and a ground truth vital sign of the person. The method further comprises training a machine-learning based feature extractor and a machine-learning based first regressor using the training dataset in a first training step. For each data record, a feature vector is extracted from the first dataset using the feature extractor, and a vital sign of the person is predicted from the feature vector using the first regressor. The first training step minimizes a difference between the predicted vital sign and the ground truth vital sign. The method further comprises training a machine-learning based embedding model and a machine-learning based second regressor using the training dataset in a second training step. For each data record, a feature vector is extracted from the first dataset using the feature extractor, an embedding vector is generated from the demographic data using the embedding model, and a vital sign of the person is predicted from the feature vector and the embedding vector using the second regressor. The second training step minimizes a difference between the predicted vital sign and the ground truth vital sign. The vital sign monitor is formed from the feature extractor, the embedding model and the second regressor.

This method trains the feature extractor, the embedding model and the second regressor of the vital sign monitor in two consecutive steps. The first step serves to train the feature extractor and a first regressor that is discarded afterwards. The second training step trains the embedding model and a second regressor which is used as the final regressor of the vital sign monitor. The two-step training process may ensure that the feature extractor learns to extract a meaningful feature vector from a first dataset comprising of a time series of a bio signal. The regressor learns to predict a vital sign from that feature vector and compensate for an effect of the demographic data. The two-stage training process allows to use different loss functions in the first training step and the second training step to optimize for different training goals in the two training steps.

In a variant of the method, the training minimizes a mean absolute error in the first training step. This may allow to train the feature extractor and the first regressor to estimate a trend of the predicted vital sign with equal contributions of each data record of the training dataset.

In a variant of the method, the training minimizes an L3 loss in the second training step. This may allow to train the second regressor to predict the vital sign accurately by penalizing large errors.

In a variant of the method, the feature extractor comprises a multi-layer perceptron, a convolutional neural network, a recurrent neural network or an attention-based model. Such architectures have proven to be effective for extracting meaningful feature vectors from temporal data.

In a variant of the method, the feature extractor comprises a LeNet architecture. This architecture may be effective in extracting a meaningful feature vector from the first dataset comprising a time series of a bio signal.

In a variant of the method, the embedding model comprises a neural network. This allows to train the embedding model to generate an embedding vector from a demographic data in a way that similar values of the demographic data are projected close to each other in the space of the embedding vector.

In a variant of the method, the second regressor comprises a neural network with a plurality of fully-connected layers. Such a regressor may be trained to predict an accurate value of the vital sign.

In a variant of the method, the second regressor comprises four fully-connected layers with 100, 50, 10 and 1 neurons. This architecture has shown to be particularly effective in predicting the vital sign from the feature vector and the embedding vector.

### BRIEF DESCRIPTION OF THE DRAWINGS

The above-described properties, features, and advantages of the invention, as well as the way in which they are achieved, will become more clearly and comprehensively understandable in connection with the following description of exemplary variants, which will be explained in more detail in connection with the drawings, in which, in schematic representation:

FIG. 1 shows a vital sign monitor;

FIG. 2 shows a first dataset comprising a time series of a bio signal;

FIG. 3 shows a training dataset;

FIG. 4 shows a first training step of a training method; and

FIG. 5 shows a second training step of the training method.

### DETAILED DESCRIPTION OF ILLUSTRATIVE EMBODIMENTS

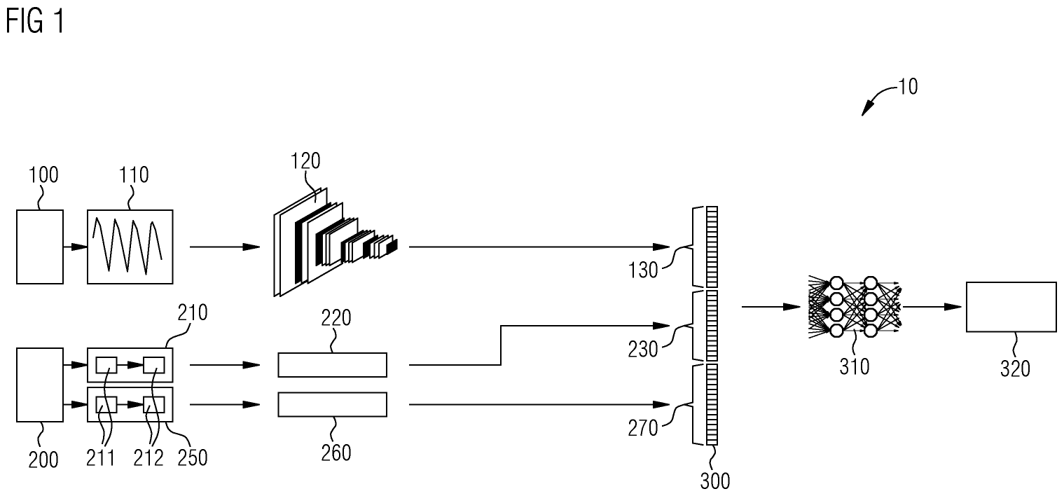

FIG. 1 shows a highly schematic depiction of a vital sign monitor 10. The vital sign monitor 10 is designed to determine or predict a vital sign 320 of a person. The vital sign 320 may be a body temperature, a heart rate, a respiratory rate, or a blood pressure, for example. The person may be a user of the vital sign monitor 10 and may also be referred to as a patient.

The vital sign monitor 10 may be integrated into a medical device or a portable device such as a smartwatch or a smartphone. In this case, the vital sign monitor 10 may be adapted to predict a vital sign 320 of the user of that device.

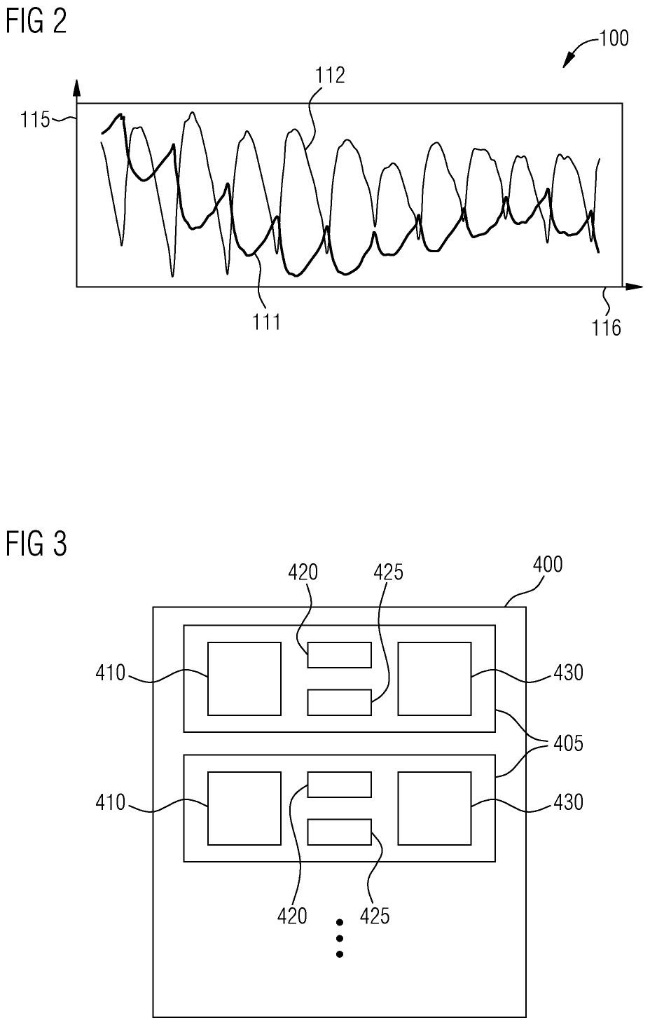

The vital sign monitor 10 comprises a sensor 100 for recording a time series of a bio signal 115 of a person as a first dataset 110. FIG. 2 shows an exemplary first dataset 110. Raw data 111 depicts a recording of the bio signal 115 over a pre-determined amount of time 116. The pre-determined amount of time may encompass several seconds, for example.

The vital sign monitor 10 may be designed to record the bio signal 115 continuously over a long period of time. In this case, the recorded data may be divided into consecutive segments of equal length that each form a first dataset 110 for one prediction of the vital sign 320. Consecutive first datasets 110 are used for consecutive predictions of the vital sign 320, allowing for a determination of a temporal trend of the vital sign 320. Each first dataset 110 may comprise 256 floating point number, for example. In this case, the bio signal 115 may recorded at 100 Hz, for example.

After recording the first dataset 110, the raw data 111 may optionally be preprocessed to transform the raw data 111 into preprocessed data 112. This is schematically shown in FIG. 2. Preprocessing the first dataset 110 may serve to remove high-frequency noise or a low-frequency modulation, for example. If preprocessing is carried out, the preprocessed data 112 is used as the first dataset 110 in the further course. Otherwise, the raw data 111 is used as the first dataset 110.

The first dataset 110 may be a photoplethysmogram, for example. In this case, the sensor 100 may be an optical sensor that includes one or more light sources and one or more light detectors. Alternatively, the first dataset 110 may be an electrocardiogram, for example. In this case, the sensor 100 may include one or more electrodes.

The first dataset 110 contains information that is meaningful for the prediction of the vital sign 320. A manual extraction of these meaningful features from the first dataset 110 may include identifying and describing relevant features and implementing a way to extract those features. Manual features extraction requires a good understanding of the background or domain. For example, a peak-to-peak time distance in a photoplethysmogram may be interpreted as the duration of a heartbeat. For an extraction of this manually identified feature, a peak detection algorithm could be applied to the first dataset 110.

An extraction of manually identified features is highly dependent on signal quality. Noise and artifacts may cause inaccuracies and errors that lead to inaccuracies of the predicted vital sign 320.

Automatic feature extraction using machine-learning based techniques can be more robust to noise. To allow for an automatic feature extraction, the vital sign monitor 10 comprises a machine-learning based feature extractor 120 for extracting a feature vector 130 from the first dataset 110. The feature vector 130 may comprise a size of 100 floating point values, for example.

The feature extractor 120 may comprise a multi-layer perceptron (MLP), a convolutional neural network (CNN), a recurrent neural network (RNN) or an attention-based model, for example. The feature extractor 120 may comprise a LeNet architecture, for example.

An interpretation of the feature vector 130 for the prediction of the vital sign 320 depends on demographic data of the person using the vital sign monitor 10. For example, men tend to have a higher blood pressure than women at similar ages. Taking demographic data of the person into account therefore may increase the accuracy of the prediction of the vital sign 320.

The vital sign monitor 10 is designed to obtain at least one demographic data 210 of the person using the vital sign monitor 10. The vital monitor 10 may comprise a data memory 200 where the demographic data 210 may be stored and from where the demographic data 210 may be obtained. The demographic data 210 may have been entered into the vital sign monitor 10 via a user interface, for example.

The vital sign monitor 10 may be designed to obtain zero, one or more further demographic data 250 in addition to the demographic data 210. The example of FIG. 1 shows one further demographic data 250.

The demographic data 210 and the further demographic data 250 may be a body height, a body mass index (BMI), an age, or a gender of the person using the vital sign monitor 10, for example.

The demographic data 210 may be a numerical value from a continuous range, for example in the case of a body height or a body weight. In this case, the demographic data 210 may be discretized using thresholds to replace an original data 211 of the demographic data 210 by a categorized demographic data 212.

In the case that the demographic data 210 is a body mass index, for example, the original data 211 may be discretized into a categorized demographic data 212 having one of five possible values, the values corresponding to an original data 211 of below 18.5, an original data 211 of between 18.5 and 24.9, an original data 211 of between 25 and 29.9, an original data 211 of between 30 and 34.9 and an original data 211 of above 35.

The same applies to any further demographic data 250.

The vital sign monitor 10 comprises an embedding model 220 for generating an embedding vector 230 from the demographic data 210. Accordingly, for each further demographic data 250, the vital sign monitor 10 respectively comprises one further embedding model 260 for generating one further embedding vector 270 from the further demographic data 250.

The following description will exemplarily describe the generation of the embedding vector 230 from the demographic data 210 using the embedding model 220. The description applies accordingly to any further embedding model 260.

The embedding vector 230 is a vector of continuous variables and may comprise between 3 and 32 elements, for example. In the case that the demographic data 210 is an age of the person, the embedding vector 230 may comprise seven elements, for example. This increased size of the embedding vector 230 compared to the demographic data 210 may help to ensure that the information contained in the demographic data 210 is taken into account for the prediction of the vital sign 320.

The embedding model 220 may transform the demographic data 210 into the space of the embedding vector 230 in a way that embedding vectors 230 corresponding to similar values or categories of the demographic data 210 are closer to each other than embedding vectors 230 corresponding to more distant values of the demographic data 210. In the case that the demographic data 210 is an age of the person, for example, an age of 20 years may be projected to a similar embedding vector 230 by the embedding model 220 as an age of 21 years.

The embedding model 220 may be learned using machine-learning techniques. This will be explained below. To this end, the embedding model 220 may comprise a neural network. The trained embedding model 220 may work like a look-up table that projects the demographic data 210 onto the embedding vector 230.

The feature vector 130, the embedding vector 230 and any further embedding vector 270 are concatenated to form a combined input vector 300. The number of elements of the combined input vector 300 corresponds to the sum of the number of elements of the feature vector 130, the embedding vector 230 and any further embedding vector 270.

The combined input vector 300 is provided to a machine-learning based regressor 310 that predicts the vital sign 320 from the combined input vector 300, hence from the feature vector 130, the embedding vector 230 and any further embedded vector 270.

The regressor 310 may comprise a neural network with a plurality of fully-connected layers, for example. In one variant, the regressor 310 may comprise four fully-connected layers with 100, 50, 10 and 1 neurons, respectively.

A production of the vital sign monitor 10 includes a training of the feature extractor 120, the embedding model 220 and any further embedding model 260, and the regressor 310.

To this end, a training dataset 400 as exemplarily shown in FIG. 3 is provided. The training dataset 400 comprises a plurality of data records 405. Each data record 405 comprises a time series of a bio signal 115 of a person as a first dataset 410, at least one demographic data 420 of the person, and a ground truth vital sign 430 of the person. In the example depicted in FIG. 3, each data record 405 comprises both a demographic data 420 and one further demographic data 425. An example of such a training dataset 400 is MIMIC-IV (Johnson, scientific data 10 (1), 1 (2023)).

As it is known to a person of skill in the art, in addition to the training dataset 400, further similar data records 405 should be kept aside for test and validation.

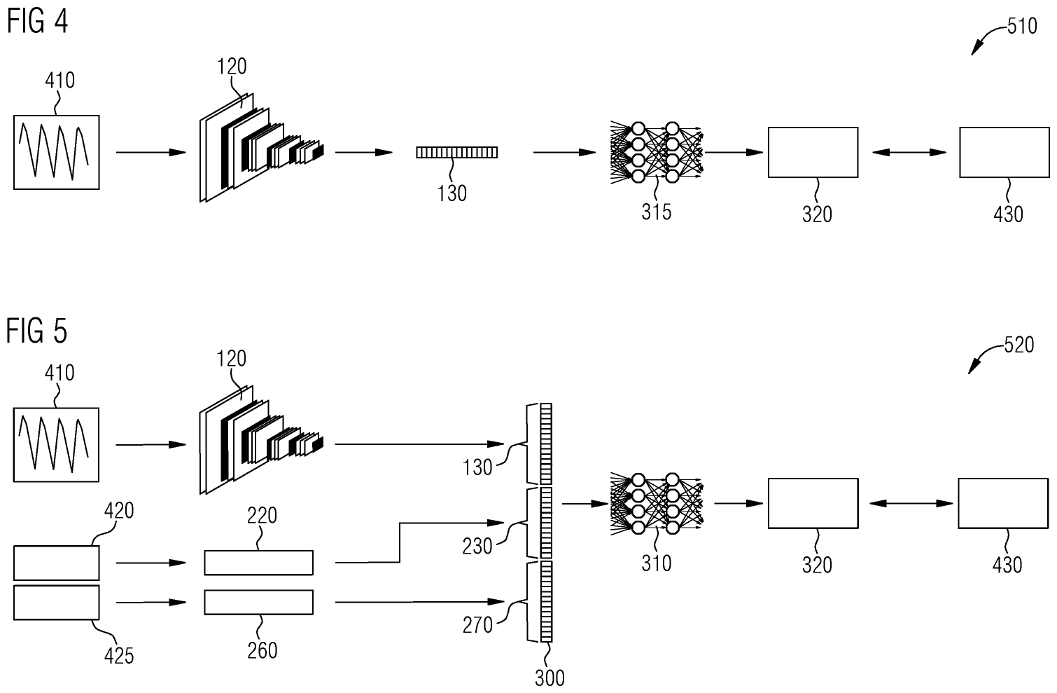

Training the machine-learning based components of the vital sign monitor 10 may be carried out in two consecutive steps. In a first training step 510 that is schematically depicted in FIG. 4, the feature extractor 120 and an intermediate regressor 315 may be trained. For each data record 405 of the training dataset 400, a feature vector 130 is extracted from the first dataset 410 using the feature extractor 120, as it has been explained above. The intermediate regressor 315 predicts a vital sign 320 from the feature vector 130. To this end, the intermediate regressor 315 is designed as the regressor 310 described above, but takes only the feature vector 130 as its input data.

The training in the first training step 510 is carried out such that a difference between the predicted vital sign 320 and the ground truth vital sign 430 is minimized on the basis of a loss function. In one variant, a mean absolute error may be used as the loss function. This may train the feature extractor 120 and the intermediate regressor 315 to correctly estimate a trend of the vital sign 320.

In a second training step 520 that is schematically illustrated in FIG. 5, the feature extractor 120 trained in the first training step 510 is used and frozen. In the second training step 520, the embedding model 220 and any further embedding model 260 is trained, as well as the final regressor 310 of the vital sign monitor 10. For each data record 405 of the training dataset 400, a feature vector 130 is extracted from the first dataset 410 using the previously trained feature extractor 120. An embedding vector 230 is generated from the demographic data 420 using the embedding model 220, as previously described above. Accordingly, further embedding vectors 270 are generated from any further demographic data 425 using the further embedding models 260.

The feature vector 130, the embedding vector 230 and any further embedding vector 270 are concatenated to form the combined input vector 300, as explained above. A vital sign 320 is predicted from the combined input vector 300 using the regressor 310, as explained above.

The training in the second step 520 is carried out to minimize a difference between the predicted vital sign 320 and the ground truth vital sign 430 of the respective data record 405 on the basis of a loss function. In the second training step 520, an L3 loss function may be used, for example. In this case, large errors of the predicted vital sign 320 are penalized.

The second training step 520 can be regarded as a calibration of the predicted vital sign 320 by considering a demographic data 210 and any further demographic data 250.

The feature extractor 120 trained in the first training step 510, the embedding model 220 and any further embedding model 260 trained in the second training step 520 and the regressor 310 trained in the second training step 520 are applied to the final vital sign monitor 10.

The invention has been illustrated and described in more detail with the aid of exemplary variants. The invention is not, however, restricted to the examples disclosed. Rather, other variants may be derived therefrom by the person skilled in the art.

What is claimed is:

1. A method for predicting a vital sign of a person,
the method comprising:
recording a time series of a bio signal of the person as a first dataset;
obtaining a demographic data of the person;
extracting a feature vector from the first dataset using a machine-learning based feature extractor;
generating an embedding vector from the demographic data using an embedding model;
predicting the vital sign of the person from the feature vector and the embedding vector using a machine-learning based regressor.

2. The method according to claim 1,
wherein the vital sign is a body temperature, a heart rate, a respiratory rate, or a blood pressure.

3. The method according to claim 1,
wherein the first dataset is a photoplethysmogram or an electrocardiogram.

4. The method according to claim 1,
wherein recording the first dataset is followed by preprocessing the first dataset to remove high-frequency noise or a low-frequency modulation.

5. The method according to claim 1,
wherein the first dataset comprises 256 floating point numbers.

6. The method according to claim 1,
wherein the demographic data is a body height, a body weight, a body mass index, an age, or a gender of the person.

7. The method according to claim 1,
wherein generating the embedding vector includes categorizing the demographic data.

8. The method according to claim 1,
wherein the embedding vector comprises between 3 and 32 elements.

9. The method according to claim 1,
wherein at least one further demographic data of the person is obtained,
wherein at least one further embedding vector is generated from the at least one further demographic data using at least one further embedding model,
wherein the vital sign of the person is predicted from the feature vector, the embedding vector, and the at least one further embedding vector.

10. A vital sign monitor comprising:
a sensor for recording the time series of the bio signal of the person as a first dataset,
wherein the vital sign monitor is adapted for obtaining a demographic data of the person,
wherein the vital sign monitor is adapted for executing the method according to claim 1.

11. The vital sign monitor according to claim 10,
wherein the vital sign monitor comprises a data memory for storing the demographic data.

12. A method for producing a vital sign monitor,
the method comprising:
providing a training dataset having a plurality of data records, wherein each data record comprises a time series of a bio signal of a person as a first dataset, a demographic data of the person, and a ground truth vital sign of the person;
training a machine-learning based feature extractor and a machine-learning based first regressor using the training dataset in a first training step,
wherein, for each data record, a feature vector is extracted from the first dataset using the feature extractor, and a vital sign of the person is predicted from the feature vector using the first regressor,
wherein the training minimizes a difference between the predicted vital sign and the ground truth vital sign;
training a machine-learning based embedding model and a machine-learning based second regressor using the training dataset in a second training step,
wherein, for each data record, a feature vector is extracted from the first dataset using the feature extractor, an embedding vector is generated from the demographic data using the embedding model, and a vital sign of the person is predicted from the feature vector and the embedding vector using the second regressor,
wherein the training minimizes a difference between the predicted vital sign and the ground truth vital sign,
wherein the vital sign monitor is formed from the feature extractor, the embedding model, and the second regressor.

13. The method according to claim 12,
wherein the training minimizes a mean absolute error in the first training step.

14. The method according to claim 12,
wherein the training minimizes an L3 loss in the second training step.

15. The method according to claim 12,
wherein the feature extractor comprises a multi-layer perceptron, a convolutional neural network, a recurrent neural network, or an attention-based model.

16. The method according to claim 12,
wherein the feature extractor comprises a LeNet architecture.

17. The method according to claim 12,
wherein the embedding model comprises a neural network.

18. The method according to claim 12,
wherein the second regressor comprises a neural network with a plurality of fully-connected layers.

19. The method according to claim 18,
wherein the second regressor comprises four fully-connected layers with 100, 50, 10 and 1 neurons.
